(19) United States
(12) Patent Application Publication
(10) Pub. No.: US 2025/0012993 A1
(43) Pub. Date: Jan. 9, 2025

(54) OPTICAL FIBER CABLE

(71) Applicant: FUJIFILM Healthcare Corporation, Kashiwa-shi (JP)

(72) Inventor: Hirozumi Takeshima, Tokyo (JP)

(21) Appl. No.: 18/750,460

(22) Filed: Jun. 21, 2024

(30) Foreign Application Priority Data
Jul. 3, 2023 (JP) 2023-109032

(51) Int. Cl. G02B 6/44; A61B 90/00; G02B 6/36

(52) U.S. Cl. CPC G02B 6/443; G02B 6/3604; A61B 90/37; A61B 2090/3782

(57) ABSTRACT

An optical fiber has one end attached to an optical connector and the other end attached to a treatment device. An optical fiber passes through a protective tube. One end of the protective tube is fixed to the optical connector, and the other end is attached with a fitting member that is fitted to a fixing member on a treatment device side. The fixing member is attached to an end part on an input side of the treatment device. Although a thin-wall portion of the fixing member is inserted into the fitting member, the fixing member and the fitting member are not fixed to each other. In a case where the treatment device rotates around an axis by an operation of a user, the optical fiber rotates in response to the rotation of the treatment device, and is twisted in the protective tube.

### CROSS REFERENCE TO RELATED APPLICATION

This application claims priority to Japanese Patent Application No. 2023-109032 filed on Jul. 3, 2023 which is incorporated herein by reference in its entirety including the specification, claims, abstract, and drawings.

### BACKGROUND OF THE DISCLOSURE

### 1. Field of the Disclosure

The present disclosure relates to an optical fiber cable.

### 2. Description of the Related Art

There are many medical devices that use an optical fiber.

For example, there is a system that improves visibility of a treatment device using a photoacoustic effect in a catheter surgery under echo guidance (see JP2019-213680A). In this system, the treatment device introduced into a body and a laser light source are connected with an optical fiber, a photoacoustic wave is generated from the treatment device using the photoacoustic effect, and the ultrasonic wave is detected by an ultrasound probe, thereby visualizing a position of the treatment device.

JP6873164B discloses a connector for connecting an optical fiber, which guides laser light to a distal end of a puncture needle, to a light source unit, for a photoacoustic effect or the like.

Here, in the catheter surgery, an operation of rotating the treatment device around an advancing direction axis or an operation of moving the treatment device is generally performed. An optical fiber for connection needs to follow such an operation. In particular, such an operation is frequently used in a guide wire, which is one type of the treatment device. In the operation of rotating the guide wire around the axis, the optical fiber is twisted and a ring or loop is formed in the middle. In a case where the treatment device is pulled in a state where a ring is formed in the middle of the optical fiber, the ring is reduced in size, and in the worst case, the optical fiber is broken.

As a technique for suppressing the twisting of the optical fiber, there is a technique disclosed in JP6492061B and JP6669898B. In JP6492061B and JP6669898B, an optical connector cable connected to a light source and a guide wire are mechanically connected with a rotatable structure such as a bearing. The optical fiber in the optical connector cable and the optical fiber in the guide wire are optically connected via a coupler.

In addition, JP2007-135947A, JP2004-065320A, JP2013-195284A, JP2016-200647A, and JP2016-507271A disclose a mechanism related to rotation or protection of an optical fiber connecting an instrument such as a probe and a main body device.

### SUMMARY OF THE DISCLOSURE

An optical fiber can be configured to be prevented from forming a ring in an optical fiber cable that connects a light source and a treatment device without using a special element such as a rotation mechanism.

An optical fiber cable disclosed in the present specification is an optical fiber cable that connects a light source and a treatment device, the optical fiber cable comprising: a connector connected to the light source; an optical fiber having one end fixed to the connector and the other end fixed to the treatment device; and a protective tube that has a bending rigidity larger than a bending rigidity of the optical fiber and covers the optical fiber from the connector to the treatment device, the protective tube being fixed to the connector but being not fixed to the treatment device, in which the protective tube and the optical fiber are not fixed to each other.

With the above-described optical fiber cable, the optical fiber can be twisted in the protective tube as the treatment device is rotated around the axis. By the twist of the optical fiber, the treatment device can be rotated without using a rotation mechanism such as a bearing.

Here, since the optical fiber is longer than the protective tube, the optical fiber may be disposed in a state of being bent in the protective tube.

With this structure, the optical fiber can be twisted in response to the rotation of the treatment device around the axis, and a gentle spiral can be formed in the protective tube.

In addition, an end part of the protective tube on a treatment device side may be fitted to a fixing member fixed to the treatment device in a rotatable manner around an axis.

In this configuration, the displacement of the protective tube in a direction perpendicular to an extending direction of the protective tube is restricted by the fixing member on the treatment device side, which is a fitting destination.

In addition, a fitting member that is fitted to the fixing member so as not to come off from the fixing member may be provided at the end part of the protective tube on the treatment device side.

In addition, an inner diameter of the protective tube may be equal to or more than 1.2 times an outer diameter of the optical fiber.

In this configuration, the optical fiber can form a spiral in the protective tube in response to the rotation of the treatment device around the axis, so that the allowable rotation count of the treatment device can be increased.

In addition, a structure may be adopted in which a part of the optical fiber cable is fixed in a state of forming a loop having a radius equal to or larger than an allowable bending radius of the optical fiber.

In this configuration, it is possible to reduce deterioration of handling of the optical fiber in a case where the optical fiber cable is made longer in order to increase the allowable rotation count of the treatment device around the axis.

According to the present disclosure, it is possible to prevent an optical fiber from forming a ring in an optical fiber cable that connects a light source and a treatment device without using a special element such as a rotation mechanism.

### BRIEF DESCRIPTION OF THE DRAWINGS

FIG. 1 is a diagram schematically showing a configuration of an embodiment of an optical fiber cable.

FIG. 2 is a diagram schematically showing a spirally twisted optical fiber in a protective tube.

FIG. 3 is a diagram schematically showing an optical fiber cable wound into a loop and fixed in a state of the loop.

FIG. 4 is a diagram schematically showing a configuration of one modification example of the optical fiber cable.

FIG. 5 is a diagram schematically showing a configuration of another embodiment of the optical fiber cable.

FIG. 6 is a diagram schematically showing a configuration of one modification example of the optical fiber cable.

FIG. 7 is a diagram schematically showing a configuration of another modification example of the optical fiber cable.

### DESCRIPTION OF THE EMBODIMENTS

Hereinafter, an embodiment (hereinafter, referred to as an embodiment) for carrying out the present disclosure will be described with reference to the drawings.

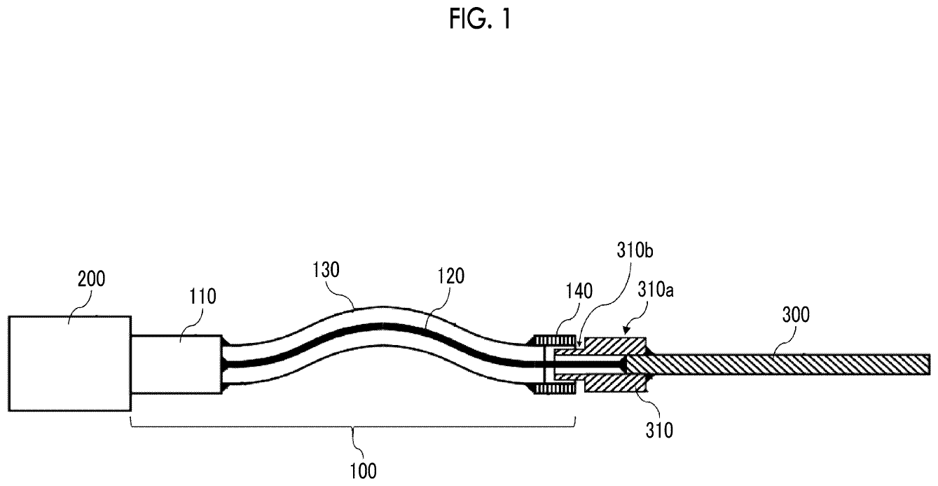

An optical fiber cable 100 illustrated in FIG. 1 comprises an optical connector 110, an optical fiber 120, a protective tube 130, and a fitting member 140. FIG. 1 and FIGS. 2 to 7 to be described below are schematic diagrams for describing features of each embodiment and each modification example, and dimensions such as lengths, widths, and diameters of members are different from actual dimensions. For example, the optical fiber cable 100 is actually much longer than the size of the optical connector 110, but is drawn to be much shorter than the actual length in order to fit within a limited size of the drawing. In addition, in the drawing, members other than a laser light source unit 200 and the optical connector 110 are represented in a schematic cross-sectional view. These schematic cross-sectional views of the members do not necessarily accurately represent actual cross sections of the members, and portions unnecessary for the description of the present embodiment are largely omitted. For example, a treatment device 300 has a specific structure such as a guide wire incorporating an optical fiber, but such a specific structure is not directly related to the features of the optical fiber cable 100 according to the embodiment. In FIG. 1, the treatment device 300 is shown as a simple rod-like member.

The optical connector 110 is a member for attachably and detachably connecting the optical fiber 120 to the laser light source unit 200.

One end part of the optical fiber 120 is attached to the optical connector 110, and the other end part is attached to the treatment device 300. The attachment of the optical fiber 120 to the treatment device 300 is a fixed attachment using an adhesive or the like. Therefore, in a case where the treatment device 300 is rotated around an axis along a extending direction (right direction in FIG. 1) of the optical fiber 120 by an operation of a user, the optical fiber 120 is also rotated by the same amount in response to the rotation. The optical fiber 120 is twisted by this rotation. In a case where the optical connector 110 is connected to the laser light source unit 200, laser light output from the laser light source unit 200 is guided to the treatment device 300 through the optical fiber 120.

The protective tube 130 is a tubular member for protecting the optical fiber 120. A length of the protective tube 130 is slightly shorter than a length of the optical fiber 120. The optical fiber 120 is accommodated in the protective tube 130 over substantially its entire length. A shape of the protective tube 130 is, for example, a cylindrical shape, and an inner diameter thereof is larger than an outer diameter of the optical fiber 120 in the protective tube 130. Therefore, there is a gap between an outer peripheral surface of the optical fiber 120 in the protective tube 130 and an inner peripheral surface of the protective tube 130. In addition, the optical fiber 120 and the protective tube 130 are not fixed to each other. Since the optical fiber 120 is longer than the protective tube 130, the optical fiber 120 may be somewhat bent or twisted in the protective tube 130.

One end part of the protective tube 130 is fixed and attached to the optical connector 110 using an adhesive or the like. The fitting member 140 that is fitted to the fixing member 310 on the treatment device 300 side is attached to the other end part of the protective tube 130. In the illustrated example, the fitting member 140 is a cylindrical member having an inner diameter that is substantially equal to an outer diameter of the protective tube 130. The attachment of the fitting member 140 to the protective tube 130 may be a fixed attachment using an adhesive or the like.

The protective tube 130 has a certain degree of flexibility such that the protective tube 130 can be bent. A material, a shape, and dimensions (for example, a tube thickness) thereof are selected such that the protective tube 130 has a higher bending rigidity than the optical fiber 120 passing through the protective tube 130. In this way, since the bending rigidity of the protective tube 130 is larger than the bending rigidity of the optical fiber 120, a ring or loop is difficult to be generated in the optical fiber 120 according to the movement of the treatment device 300.

The protective tube 130 may be formed of, for example, a material having a low friction coefficient, such as Teflon (registered trademark) or a polyester resin. As a result, the friction between the optical fiber 120 and the protective tube 130, and the friction in the protective tube 130 (for example, between one part and another part of one protective tube 130) are reduced, and the optical fiber cable 100 can be easily handled.

Although the protective tube 130 has a thickness, for the sake of simplicity, the protective tube 130 is represented by one thick line in the drawing.

The treatment device 300 is an example of a device that uses the laser light supplied via the optical fiber 120, and performs an action for treatment or the like on a patient. The treatment device 300 may be a device that treats an affected part with laser light, or may be a device that performs an action of assisting the treatment, such as illuminating a body cavity or the like with laser light or generating an ultrasonic wave by a photoacoustic effect.

Although it is merely an example, the treatment device 300 may be a guide wire used in a catheter surgery or the like. As an example of such a guide wire, there is a guide wire comprising a generation member that generates a photoacoustic signal at a distal end (that is, a left end of the treatment device 300 in the drawing). This type of guide wire incorporates a light guide member such as an optical fiber that guides light from an input end (that is, a right end of the treatment device 300 in the drawing) to the distal end. The input end of the member is fixedly connected (for example, adhered) to the optical fiber 120 in the optical fiber cable 100 in a manner that the laser light can be transmitted therethrough. Alternatively, the optical fiber 120 itself may extend through the guide wire to the photoacoustic generating member provided at the distal end of the guide wire. In this example, a pulse of the laser light emitted from the laser light source unit 200 passes through the optical fiber 120 in the optical fiber cable 100 and the light guide member in the guide wire, and is emitted to the photoacoustic generating member at the distal end of the guide wire. By the emission of the pulsed laser light, the photoacoustic generating member expands and contracts rapidly, and thus the ultrasonic wave is generated. An ultrasound equipment (not shown) detects the ultrasonic wave and forms an image of the detected wave.

The fixing member 310 is fixedly attached to the input end (that is, the right end in the drawing) of the treatment device 300 using an adhesive or the like. The fixing member 310 connects the protective tube 130 and the treatment device 300 to each other by being fitted to the fitting member 140 of the optical fiber cable 100. The fixing member 310 may be used to fix the optical fiber 120 to the treatment device 300 side. For example, the optical fiber 120 can be fixed to the treatment device 300 via the fixing member 310 by adhering a side surface of the end part (that is, the left end in the drawing) of the optical fiber 120 to an inner surface of the fixing member 310.

The optical fiber 120 extends from the optical connector 110 to the treatment device 300 through the protective tube 130 and the fixing member 310.

Although it is merely an example, in the illustrated example, the fixing member 310 is a cylindrical member having an inner diameter that is substantially equal to an outer diameter of the treatment device 300, and is composed of a thick-wall portion 310a and a thin-wall portion 310b. An outer diameter of the thin-wall portion 310b is equal to or smaller than an inner diameter of the fitting member 140. The thin-wall portion 310b is inserted into the fitting member 140 at the end part of the protective tube 130, so that the fixing member 310 and the fitting member 140 are fitted to each other. In addition, since an outer diameter of the thick-wall portion 310a is larger than the inner diameter of the fitting member 140, only the thin-wall portion 310b of the fixing member 310 can enter the fitting member 140.

Lengths of the optical fiber 120, the protective tube 130, the fitting member 140, and the thin-wall portion 310b are determined such that the thin-wall portion 310b does not come off from the fitting member 140 in a state where the treatment device 300 is pulled and the optical fiber 120 is straight without bending.

Typically, there is a gap between an outer surface of the thin-wall portion 310b and an inner surface of the fitting member 140. Therefore, the fixing member 310 and the treatment device 300 fixedly connected to the fixing member 310 are rotatable with respect to the fitting member 140 and the protective tube 130 fixedly connected to the fitting member 140. Processing or design for reducing the friction between the outer surface of the thin-wall portion 310b and the inner surface of the fitting member 140 may be adopted. For example, a lubricant may be applied between both surfaces, or both surfaces or at least one surface may be subjected to processing for reducing friction such as Teflon processing. In addition, for example, in a case where a member for reducing friction, such as a Teflon bush, is mounted to the thin-wall portion 310b, the friction between the thin-wall portion 310b side and the fitting member 140 may be reduced. Such a member for reducing friction may be provided on the inner surface of the fitting member 140, or may be provided on both the outer surface of the thin-wall portion 310b and the inner surface of the fitting member 140. In addition, the thin-wall portion 310b and the fitting member 140 may be made of a material having a low friction coefficient.

In addition, as another example, the fixing member 310 and the fitting member 140 may be fitted in a state where the outer surface of the thin-wall portion 310b and the inner surface of the fitting member 140 are in contact with each other, but even in this case, the fixing member 310 is rotatable with respect to the fitting member 140 in order not to hinder the operation of the treatment device 300. In this regard, it is sufficient to select the materials of the fixing member 310 and the fitting member 140 such that the friction between the two surfaces is reduced to some extent, or to adopt processing or design (for example, lubrication or adoption of a member for reducing friction) for reducing the friction between the two surfaces.

The connection structure between the optical fiber cable 100 and the treatment device 300 has been described above. In this structure, the optical fiber 120 is somewhat longer than the protective tube 130. Therefore, the optical fiber 120 is disposed in a state of being somewhat bent in the protective tube 130 in a state where the fitting member 140 of the optical fiber cable 100 and the fixing member 310 on the treatment device 300 side are fitted to each other. In addition, in a case where the treatment device 300 is rotated around the axis, the optical fiber 120 that is bent in the protective tube 130 is twisted in the protective tube 130 in response to the rotation, and a gentle spiral is formed in the protective tube 130 as the twist is strong.

Here, since the optical fiber 120 is fixedly attached to the treatment device 300, in a case where the treatment device 300 is rotated about the axis in a longitudinal direction (that is, in the drawing, in a direction from the end part on the side connected to the optical fiber 120 of the treatment device 300 to the end part on the opposite side) by the operation of the user, the fixed end of the optical fiber 120 is rotated by the same amount. By this rotation, a twisting force around the axis acts on the optical fiber 120. On the other hand, since the protective tube 130 is not fixed to the treatment device 300, even in a case where the treatment device 300 is rotated around the axis, the protective tube 130 does not follow the rotation of the treatment device 300.

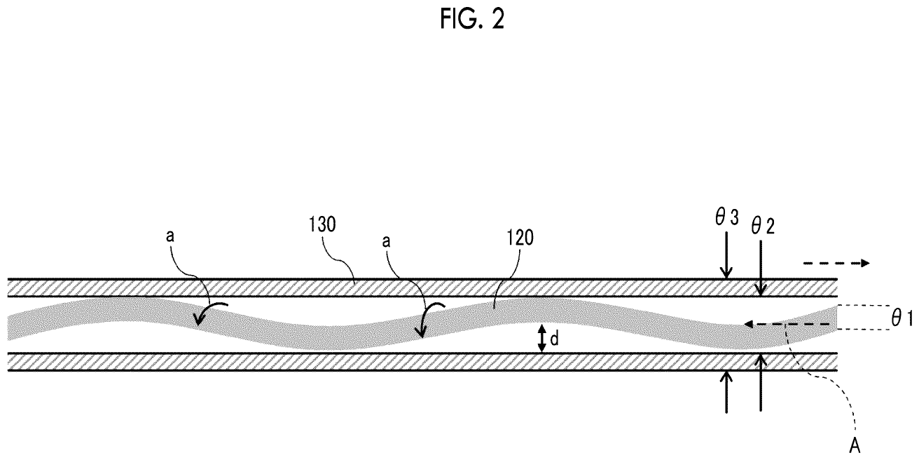

In a case where the treatment device 300 is rotated around the axis, as shown in FIG. 2, the optical fiber 120 receives a twisting force in a direction of an arrow a by the rotation of the treatment device 300, and is twisted in the protective tube 130. On the other hand, the protective tube 130 and the fitting member 140 at the end part of the protective tube 130 are not in contact with the fixing member 310 on the treatment device 300 side, or even in a case where the protective tube 130 and the fitting member 140 are in contact with the fixing member 310, the friction with the inner surface of the fixing member 310 is sufficiently small. Therefore, even in a case where the treatment device 300 is rotated around the axis, the protective tube 130 is not affected at all or is hardly affected by the rotation of the treatment device 300, and is not twisted at all or is hardly twisted. In other words, in a case where the treatment device 300 is rotated around the axis, the protective tube 130 is not at all or substantially not resistant, and only the optical fiber 120 is substantially resistant. In a case where the optical fiber 120 is made sufficiently long (for example, 2 m or more), a torsional spring property of the optical fiber 120, which is a resistance in a case where the treatment device 300 is rotated around the axis, can be made sufficiently small, and operability of the treatment device 300 is hardly affected.

In a case where the twist is large to some extent, the optical fiber 120 forms a loose spiral in the protective tube 130. By the formation of the spiral, the optical fiber 120 receives a force in a direction of an arrow A in the drawing, and the length of the optical fiber 120 along a direction in which the optical fiber cable 100 extends is shortened. In this case as well, the optical fiber 120 is made longer than the protective tube 130 to some extent, whereby it is possible to prevent the fixing member 310 on the treatment device 300 side from being strongly pressed against the fitting member 140 on the protective tube 130 side even in a case where the optical fiber 120 is shortened. In a case where the fixing member 310 is strongly pressed against the fitting member 140, a resistance to the operation of rotating the treatment device 300 around the axis is increased because of the friction between the fixing member 310 and the fitting member 140.

FIG. 2 is an emphasized diagram for description, and the dimensions are not actual. For example, a relationship between a diameter φ1 of the optical fiber 120 and an inner diameter φ2 and an outer diameter φ3 of the protective tube 130, which are shown in the drawing, does not necessarily match the actual relationship. In addition, in FIG. 2, a pitch of the spiral formed by the twisting of the optical fiber 120 is drawn to be emphasized and shorter than the actual pitch.

In a case where the treatment device 300 is a guide wire for the catheter surgery, an outer diameter of the guide wire is around 0.5 mm (for example, about 0.3 to 1 mm), and a diameter of the optical fiber that guides light to the photoacoustic generating member at the distal end through the guide wire is, for example, about 0.2 mm. A diameter of the optical fiber 120 that guides the laser light to the guide wire is also, for example, about 0.2 mm. In addition, in this case, a length of the optical fiber cable 100 (that is, the optical fiber 120 and the protective tube 130) is preferably 2 m or more, and more preferably about 3 m. In a case where the optical fiber 120 whose diameter is about 0.2 mm is used and the optical fiber 120 has a length of 2 m or more, the torsional spring property that resists the operation of rotating the treatment device 300 around the axis can be made sufficiently small.

The inner diameter 2 of the protective tube 130 is determined such that a sufficient gap is formed between the optical fiber 120 and the inner surface of the protective tube 130. The sufficient gap referred to herein is a gap equal to or larger than a degree to which the optical fiber 120 inserted into the protective tube 130 can be freely rotated. In order to satisfy such a condition, it is desirable that the inner diameter φ2 of the protective tube 130 is equal to or more than 1.2 times the diameter φ1 of the optical fiber 120. In a case where the protective tube 130 having such an inner diameter φ2 is used as the protective tube 130, the optical fiber 120 can be twisted spirally in the protective tube 130.

As described above, in the present embodiment, the protective tube 130 and the optical fiber 120 have a dimensional relationship that allows the optical fiber 120 to be twisted spirally in the protective tube 130. Accordingly, it is possible for the treatment device 300 to rotate more around the axis than in a case where the protective tube 130 and the optical fiber 120 have a dimensional relationship in which the optical fiber 120 cannot form a spiral in the protective tube 130. This increases a degree of freedom of the operation of the treatment device 300.

For example, it is assumed that the diameter φ1 of the optical fiber 120 is about 0.2 mm, the inner diameter φ2 of the protective tube 130 is about 0.4 mm, the total length of the optical fiber 120 and the protective tube 130 is about 3 m, and the optical fiber 120 forms a spiral twisted about 100 times within the total length. In this case, a curvature radius of the spiral is about 20 cm. The curvature radius is sufficiently larger than an allowable bending radius of the optical fiber 120 determined by the standard, which is 30 mm.

In addition, the inner diameter φ2 of the protective tube 130 is made smaller than the diameter of the thick-wall portion 310a of the fixing member 310 such that the fixing member 310 does not enter the protective tube 130.

In addition, the material, the inner diameter, and the outer diameter of the protective tube 130 are determined such that the bending rigidity of the protective tube 130 is larger than the bending rigidity of the optical fiber 120, so that the protective tube 130 does not form a ring or loop together with the optical fiber 120 in a case where the optical fiber 120 is strongly twisted. However, in a case where the bending rigidity of the protective tube 130 is too large, there is a risk that the operation of the treatment device 300 is hindered, or a difference in behavior between the protective tube 130 and the optical fiber 120 is too large, which may cause damage to the optical fiber 120. In order to avoid such a problem, it is necessary to prevent the bending rigidity of the protective tube 130 from being too large. In addition, for the purpose of restricting the deformation of the optical fiber 120, it is desirable that the protective tube 130 has a smaller diameter. From the viewpoint of these conditions and manufacturing, it is desirable that the inner diameter φ2 of the optical fiber 120 is about, for example, 1.2 times to 4 times the diameter φ1 of the optical fiber 120, and the outer diameter φ3 is about, for example, 1.5 times to 3 times the inner diameter φ2.

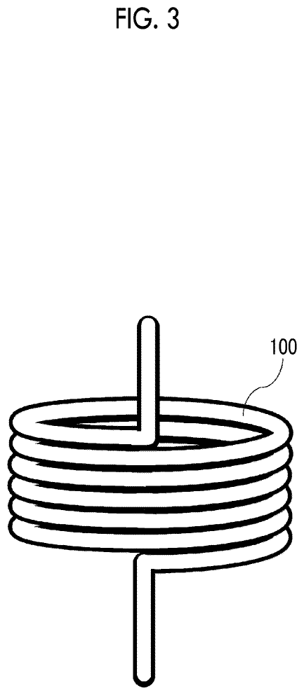

In a case where the optical fiber cable 100 is made long (for example, 2 m or more), there is a risk that the handling may deteriorate, such as being caught on other cables or other objects. In order to alleviate such a problem, the optical fiber cable 100 may be wound in a loop shape having a radius equal to or larger than the allowable bending radius (for example, 30 mm or 60 mm) of the optical fiber 120, as shown in FIG. 3, and the loop may be fixed by adhesion, binding using a binding band, or the like. Since this loop is fixed, even in a case where the treatment device 300 is pulled, the radius of the loop is not reduced, and there is little risk of deterioration in the characteristics of the optical fiber 120 or breakage of the optical fiber 120.

In addition, in the embodiment of FIG. 1, in order to prevent the fitting member 140 from coming off from the fixing member 310, the fitting member 140 and the fixing member 310 may have a coming-off prevention structure. For example, a flange is formed at an end part of the thin-wall portion 310b of the fixing member 310 on the protective tube 130 side, and an end part of the fitting member 140 on the treatment device 300 side is narrowed to have an opening smaller than a diameter of the flange.

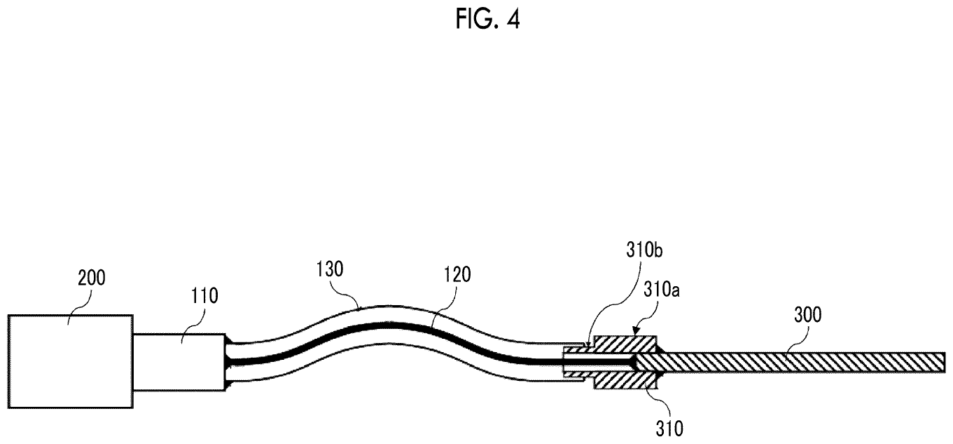

FIG. 4 shows a modification example of the embodiment shown in FIG. 1. In FIG. 4, the same components as the components shown in FIG. 1 are denoted by the same reference numerals as those in FIG. 1, and the redundant description will be omitted.

In this modification example, the fitting member 140 is removed from the embodiment shown in FIG. 1, and a structure is adopted in which the thin-wall portion 310b of the fixing member 310 is directly inserted into the protective tube 130. In this structure, the outer diameter of the thin-wall portion 310b is equal to or smaller than the inner diameter of the protective tube 130, and the outer diameter of the thick-wall portion 310a is larger than the inner diameter of the protective tube 130.

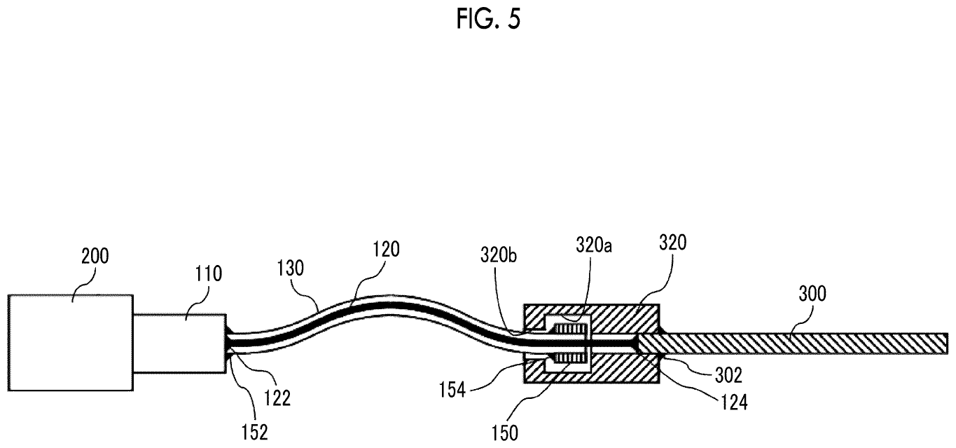

Next, another embodiment will be described with reference to FIG. 5. In FIG. 5, the same components as the components shown in FIG. 1 are denoted by the same reference numerals as those in FIG. 1, and the redundant description will be omitted.

In the embodiment of FIG. 1 described above, a structure is adopted in which the thin-wall portion 310b of the fixing member 310 on the treatment device 300 side enters the fitting member 140 at the end part of the protective tube 130. On the other hand, in the embodiment of FIG. 5, on the contrary, a structure is adopted in which a fitting member 150 at the end part of the protective tube 130 enters a fixing member 320 on the treatment device 300 side.

In the embodiment of FIG. 5, one end part of the optical fiber 120 is fixed to the optical connector 110 using an adhesive 122, and the other end part is fixed to the treatment device 300 using an adhesive 124. In addition, in the protective tube 130 into which the optical fiber 120 is inserted, one end part thereof is fixed to the optical connector 110 using an adhesive 152. In addition, the fitting member 150 is fixedly attached to the other end part of the protective tube 130 using an adhesive 154. The inner diameter of the fitting member 150 is larger than the diameter of the optical fiber 120 and is substantially the same as, for example, the inner diameter of the protective tube 130. The outer diameter of the fitting member 150 is larger than the outer diameter of the protective tube 130.

The fixing member 320 is a substantially tubular member, and one end part thereof is fixedly attached to the treatment device 300 using an adhesive 302. An accommodation chamber 320a that accommodates the fitting member 150 is formed inside the fixing member 320. The accommodation chamber 320a is a cylindrical space, and an inner diameter thereof is larger than the outer diameter of the fitting member 150. The other end part of the fixing member 320, that is, the end part on the protective tube 130 side has a structure in which an opening 320b having a diameter smaller than the outer diameter of the fitting member 150 is formed. Therefore, the fitting member 150 does not come off from the inside of the accommodation chamber 320a to the optical connector 110 side. That is, with this structure, the treatment device 300 is prevented from coming off from the protective tube 130.

A length of the accommodation chamber 320a in a direction in which the treatment device 300 extends (horizontal direction in the drawing) is longer than the length of the fitting member 150 in the same direction. Therefore, the treatment device 300 can be moved forward and backward with respect to the protective tube 130 to some extent while maintaining a state in which the fitting member 150 is fitted to the fixing member 320. In addition, the difference in length is useful for absorbing an influence of the shortening of the optical fiber 120 in a case where the optical fiber 120 is twisted to form a spiral by the rotation of the treatment device 300 around the axis.

The portions other than the shape and the structure of the fixing member 320 and the fitting member 150 in this embodiment may be the same as those in FIG. 1.

In the example of FIG. 5, the optical fiber 120 is attached to the optical connector 110 and the treatment device 300 using the adhesives 122 and 124, but this is merely an example. Any attachment method may be used as long as the two are fixed to each other. Similarly, a fixing method other than the adhesive may be used for attaching the optical connector 110 and the fitting member 150 to the protective tube 130, and for attaching the fixing member 320 to the treatment device 300.

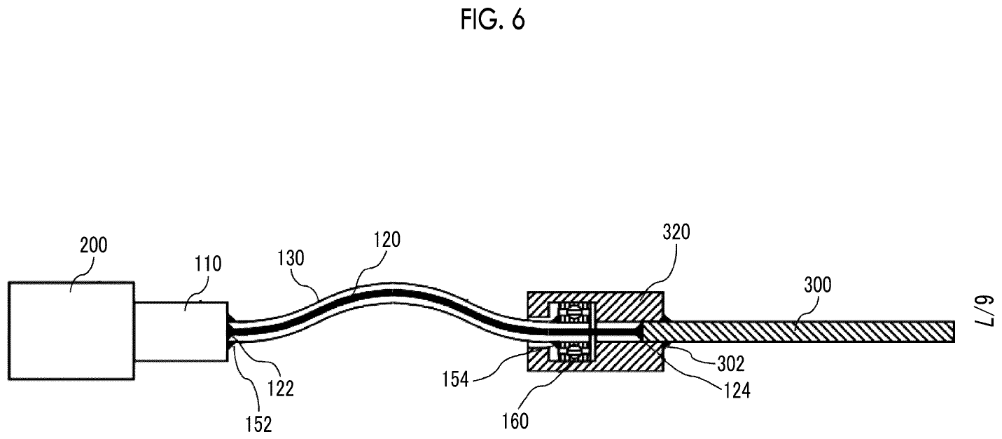

FIG. 6 shows one modification example of the embodiment of FIG. 5. In FIG. 6, the same components as the components shown in FIGS. 1 and 5 are denoted by the same reference numerals as those in FIG. 1, and the redundant description will be omitted.

In the modification example of FIG. 6, the fitting member 150 in the embodiment of FIG. 5 is replaced with a bearing 160. An outer diameter of the bearing 160 is substantially the same as an inner diameter of the accommodation chamber 320a of the fixing member 320, and the two are fitted together with substantially no gap. An outer peripheral surface of the bearing 160 may or may not be fixed to a cylindrical inner peripheral surface of the accommodation chamber 320a. In a case where the outer peripheral surface of the bearing 160 is not fixed, a material of the outer peripheral surface of the bearing 160 and a material of the inner peripheral surface of the accommodation chamber 320a may be selected, or processing or design for reducing friction between the two surfaces may be adopted so that the outer peripheral surface of the bearing 160 is slidable in an axial direction with respect to the inner peripheral surface of the accommodation chamber 320a.

The protective tube 130 is fixedly attached to an inner ring of the bearing 160 using an adhesive or the like.

In this modification example, the rotation of the treatment device 300 around the axis is smoothed by the bearing 160. In addition, since the bearing 160 is fitted to the inner peripheral surface of the accommodation chamber 320a with substantially no gap, the protective tube 130 does not wobble with respect to the fixing member 320 in a radial direction of the bearing 160, and the treatment device 300 can be easily handled.

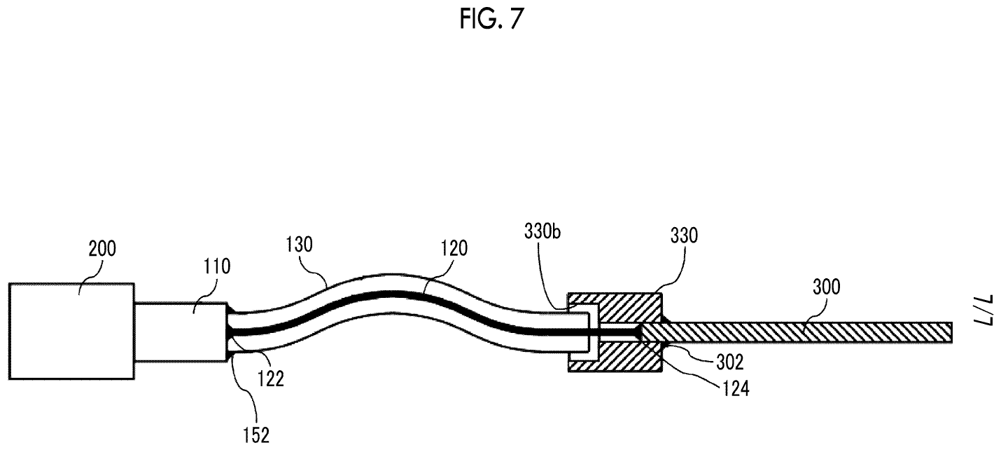

FIG. 7 shows another modification example. In FIG. 7, the same components as the components shown in FIGS. 1 and 5 are denoted by the same reference numerals as those in FIG. 1, and the redundant description will be omitted.

In this modification example, the fitting member 150 is removed from the embodiment shown in FIG. 5, and a structure is adopted in which the protective tube 130 is directly inserted into an opening portion 330b of the fixing member 330. An inner diameter of the opening portion 330b is equal to or larger than the outer diameter of the protective tube 130. There may be a gap between an outer peripheral surface of the protective tube 130 and an inner peripheral surface of the opening portion 330b. In addition, the outer peripheral surface of the protective tube 130 may be fitted to the inner peripheral surface of the opening portion 330b with substantially no gap. Note that, even in this case, the materials of the protective tube 130 and the fixing member 330 are selected, or processing or design for reducing the friction is performed such that the friction between the outer peripheral surface of the protective tube 130 and the inner side surface of the opening portion 330b is sufficiently small, so that the operation of rotating the treatment device 300 around the axis is not hindered.

In this structure, the fixing member 330 is fixed in position by being inserted into the end part of the protective tube 130, and is pulled by the optical fiber 120 toward the laser light source unit 200 side, so that the fixing member 330 is not separated from the protective tube 130.

In each of the embodiments and the modification examples described above, the optical fiber 120 is not limited to a single line and may be a combination of a plurality of optical fibers.

In the above, the guide wire using the photoacoustic effect has been described as a main example as the treatment device 300, but the optical fiber cable 100 according to each of the above-described embodiments and the modification examples can also be applied to another type of treatment device 300 that uses light supplied via the optical fiber 120.

In addition, the optical fiber cable 100 can also be applied to the treatment device 300 including an optical attachment/detachment portion or an extension cable. In this case, an end part of the optical fiber 120 in the optical fiber cable 100 is fixedly connected to an end part of the optical attachment/detachment portion or the extension cable included in the treatment device 300. An end part of the protective tube 130 is not fixed to the end part of the optical attachment/detachment portion or the extension cable. A body portion of the treatment device 300 is attachable to and detachable from the attachment/detachment portion or the extension cable. In this example, since the attachment/detachment portion or the extension cable is a part of the treatment device 300, it can be said that the optical fiber 120 is fixed to the treatment device 300. In this example, as the optical attachment/detachment portion, for example, a configuration in which two optical fibers can be attachably and detachably connected via the coupler, as shown in JP6492061B and JP6669898B, may be adopted. In the device disclosed in JP6492061B and JP6669898B, the attachment/detachment portion having the coupler has a rotatable structure such as a bearing, and the rotation of the guide wire around the axis is allowed by this structure. On the other hand, in the present embodiment, even in a case where the attachment/detachment portion does not have such a rotatable structure, the influence of the rotation of the treatment device 300 around the axis can be absorbed by the twist of the optical fiber 120.

The embodiments and the modification examples of the present disclosure have been described above. These embodiments and modification examples are merely examples for description. Various modifications and improvements can be made within the scope of the present disclosure.

What is claimed is:

1. An optical fiber cable that connects a light source and a treatment device, the optical fiber cable comprising:
a connector connected to the light source;
an optical fiber having one end fixed to the connector and the other end fixed to the treatment device; and
a protective tube that has a bending rigidity larger than a bending rigidity of the optical fiber and covers the optical fiber from the connector to the treatment device, the protective tube being fixed to the connector but being not fixed to the treatment device,
wherein the protective tube and the optical fiber are not fixed to each other.

2. The optical fiber cable according to claim 1,
wherein, since the optical fiber is longer than the protective tube, the optical fiber is disposed in a state of being bent in the protective tube.

3. The optical fiber cable according to claim 1,
wherein an end part of the protective tube on a treatment device side is fitted to a fixing member fixed to the treatment device in a rotatable manner around an axis.

4. The optical fiber cable according to claim 3,
wherein a fitting member that is fitted to the fixing member so as not to come off from the fixing member is provided at the end part of the protective tube on the treatment device side.

5. The optical fiber cable according to claim 1,
wherein an inner diameter of the protective tube is equal to or more than 1.2 times an outer diameter of the optical fiber.

6. The optical fiber cable according to claim 1,
wherein a part of the optical fiber cable is fixed in a state of forming a loop having a radius equal to or larger than an allowable bending radius of the optical fiber.
